(19) United States
(12) Patent Application Publication
(10) Pub. No.: US 2006/0018957 A1
(43) Pub. Date: Jan. 26, 2006

(54) Pharmaceutical dosage forms including rasagiline

(76) Inventors: E. Itzhak Lerner, Petach Tikva (IL); Vered Rosenberger, Mudiin (IL); Ofer Aqua, Ofra (IL); Moshe Flashner-Barak, Petach Tikva (IL); Adrian Gilbert, Ra'anana (IL); Daniella Licht, Givat Shmuel (IL); Shulamit Patashnik, Reut (IL)

Correspondence Address: COOPER & DUNHAM, LLP, 1185 AVENUE OF THE AMERICAS, NEW YORK, NY, 10036, US

(21) Appl. No.: 11/190,623

(22) Filed: Jul. 26, 2005

Related U.S. Application Data
(60) Provisional application No. 60/591,359, filed on Jul. 26, 2004.
(60) Provisional application No. 60/606,241, filed on Sep. 1, 2004.

(51) Int. Cl. A61K 9/48; A61K 9/22

(52) U.S. Cl. 424/451; 424/468

(57) ABSTRACT

Provided are pharmaceutical dosage forms that include rasagiline formulated and fabricated so that release of rasagiline in the stomach is retarded or inhibited.

This application claims benefit of U.S. Provisional Application No. 60/591,359, filed Jul. 26, 2004, and U.S. Provisional Application No. 60/606,241, filed Aug. 31, 2004, the contents of both of which are hereby incorporated by reference.

Throughout this application various publications, published patent applications and published patents are referenced. The disclosures of these publications in their entireties are hereby incorporated by reference into this application in order to more fully describe the state of the art to which this invention pertains.

### BACKGROUND OF THE INVENTION

U.S. Pat. Nos. 5,532,415, 5,387,612, 5,453,446, 5,457,133, 5,599,991, 5,744,500, 5,891,923, 5,668,181, 5,576,353, 5,519,061, 5,786,390, 6,316,504, 6,630,514 disclose R(+)-N-propargyl-1-aminoindan (“R-PAI”), also known as rasagiline. Rasagiline has been reported to be a selective inhibitor of the B-form of the enzyme monoamine oxidase (“MAO-B”) and is useful in treating Parkinson's disease and various other conditions by inhibition of MAO-B in the brain.

U.S. Pat. No. 6,126,968 and PCT publication WO 95/11016, hereby incorporated by reference, disclose pharmaceutical compositions comprising rasagiline.

A concern in using monoamine oxidase (“MAO”) inhibitors is the risk of hypertensive crises, often called the “cheese effect.” (Simpson, G. M. and White K. “Tyramine studies and the safety of MAOI drugs.” J Clin Psychiatry. 1984 July; 45 (7 pt 2): 59-91.) This effect is caused by inhibition of peripheral MAO. A high concentration of peripheral MAO is found in the stomach.

A further concern in Parkinson's disease patients is that many patients suffer from delayed gastric emptying (Pfeiffer, R. F. and Quigley, E. M. M. “Gastrointestinal motility problems in patients with Parkinson's disease: Epidemiology, pathophysiology, and guidelines for management,” CNS-Drugs, 1999, 11(6): 435-448; Jost, W. H., “Gastrointestinal motility problems in patients with Parkinson's disease: Effects of antiparkinsonian treatment and guidelines for management”, Drugs and Aging, 1997, 10(4): 249-258). Delayed gastric emptying (prolonged gastric residence) can be a cause of increased inhibition of peripheral MAO, and can contribute to the cheese effect.

MAO inhibitors that selectively inhibit MAO-B are largely devoid of the potential to cause cheese effect. Nonetheless, the possibility exists that delayed gastric emptying of R-PAI may contribute to the cheese effect.

### SUMMARY OF THE INVENTION

The present inventions provides an oral pharmaceutical dosage form comprising rasagiline and adapted to retard or inhibit the release of rasagiline in the stomach.

### BRIEF DESCRIPTION OF THE FIGURES

FIG. 1 Sectional perspective, side and top down view of a solid dosage form with a recessed core tablet of active ingredient in a compressed annular body of powder or granular material in accordance with the invention.

### DETAILED DESCRIPTION OF THE INVENTION

The present invention provides an oral pharmaceutical dosage form comprising rasagiline and adapted to retard or inhibit the release of rasagiline in the stomach.

In a further embodiment, the oral pharmaceutical dosage form is a tablet, a capsule, or a core sheathed in an annular body.

In a further embodiment, the pharmaceutical dosage form is a tablet.

In a further embodiment, the pharmaceutical dosage form comprises an enteric coating.

In a further embodiment, the pharmaceutical dosage form is a core sheathed in an annular body.

In a further embodiment, the core is a tablet.

In a further embodiment, the annular body is an annular sheath.

In a further embodiment, the core has an enteric coating.

In a further embodiment, the pharmaceutical dosage form is a capsule.

In a further embodiment, the capsule comprises solid inactive particles.

In a further embodiment, the particles are spheres, microparticles, nanoparticles or pellets made by spheronization, or a mixture thereof.

In a further embodiment, the particles are spheres.

In a further embodiment, the particles are comprised of sugars, alcohols, polyols or celluloses, or mixtures thereof.

In a further embodiment, the particles are comprised of sugars.

In a further embodiment, the solid inactive particles comprise rasagiline.

In a further embodiment, the particles are coated with rasagiline.

In a further embodiment, the particles comprise an enteric coating.

In a further embodiment, the enteric coating comprises polymeric methacrylate.

In a further embodiment, the polymeric methacrylate is methacrylic acid-ethyl acrylate copolymer (1:1) dispersion 30 percent.

In a further embodiment, the enteric coating further comprises a plasticizer.

In a further embodiment, the plasticizer is triethyl citrate.

In a further embodiment, the oral pharmaceutical dosage form, upon administration to a patient, releases rasagiline substantially in the intestinal tract.

In a further embodiment, the rasagiline is released substantially in the small intestine.

In a further embodiment, the pharmaceutical dosage form has a rasagiline release profile in a United States Pharmacopoeia Apparatus II of less than 10% dissolution in 500 ml 0.1N HCl at 37° C. and 50 rpm after 3 hours, and more than 90% dissolution in phosphate buffer at a pH of 6.8 after an additional 2 hours.

In a further embodiment, the pharmaceutical dosage form has a rasagiline release profile in a United States Pharmacopoeia Apparatus II of less than 5% dissolution in 500 ml 0.1N HCl at 37° C. and 50 rpm after 3 hours, and more than 95% dissolution in phosphate buffer at a pH of 6.8 after an additional 2 hours.

In a further embodiment, the pharmaceutical dosage form has a rasagiline release profile in a United States Pharmacopoeia Apparatus II of less than 3% dissolution in 500 ml 0.1N HCl at 37° C. and 50 rpm after 3 hours, and more than 97% dissolution in phosphate buffer at a pH of 6.8 after an additional 2 hours.

In a further embodiment, the pharmaceutical dosage form has a rasagiline release profile in a United States Pharmacopoeia Apparatus II of less than 2% dissolution in 500 ml 0.1N HCl at 37° C. and 50 rpm after 3 hours, and more than 98% dissolution in phosphate buffer at a pH of 6.8 after an additional 2 hours.

In a further embodiment, the pharmaceutical dosage form has a rasagiline release profile in a United States Pharmacopoeia Apparatus II of less than 1% dissolution in 500 ml 0.1N HCl at 37° C. and 50 rpm after 3 hours, and more than 99% dissolution in phosphate buffer at a pH of 6.8 after an additional 2 hours.

In a further embodiment, the present invention provides a method of treating a patient suffering from Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, or an affective illness, comprising administering to the patient the oral dosage form.

In a further embodiment, the present invention provides a method for treating a patient suffering from Parkinson's disease.

In a further embodiment, the patient presents impaired gastric motility.

In a further embodiment, the present invention provides the use of the pharmaceutical dosage form for the preparation of a medicament for the treatment of a patient suffering from Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, or an affective illness.

In a further embodiment, the present invention provides the use for the treatment of a patient suffering from Parkinson's disease.

In a further embodiment, the patient presents impaired gastric motility.

In a further embodiment, the present invention provides the pharmaceutical dosage form for use in treating a patient suffering from Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, or an affective illness.

In a further embodiment, the present invention provides the pharmaceutical dosage form for use in treating a patient suffering from Parkinson's disease.

In a further embodiment, the patient presents impaired gastric motility.

The instant invention provides a solution to the problem of peripheral MAO inhibition by providing pharmaceutical dosage forms comprising rasagiline which are adapted to inhibit the release or absorption of rasagiline in the stomach (i.e. delay the release of rasagiline until at least a portion of the dosage form has traversed the stomach). This avoids or minimizes absorption of rasagiline in the stomach, thereby avoiding or minimizing the potential cheese effect.

The pharmaceutical dosage form may be comprised of an acid resistant excipient which prevents the dosage form or parts thereof from contacting the acidic environment of the stomach. The acid resistant excipient may coat the rasagiline in the form of an enteric coated tablet, capsule, or gelatin capsule. Enteric coating, in the context of this invention, is a coating which prevents the dissolution of an active ingredient in the stomach. This is determined by measuring the dissolution of the pharmaceutical dosage form in acidic solution, as defined by USP methods. Even in enteric pharmaceutical dosage forms, some of the dosage form may dissolve in the stomach; however, the dosage form may still be considered enteric according to USP standards.

A specific example of enteric coating is a polymeric methacrylate, most especially where the polymeric methacrylate is methacrylic acid-co-ethyl acrylate (1:1). The enteric coating can contain a plasticizer, e.g. triethyl citrate. Enteric coatings are generally described in, e.g., United States Pharmacopeia, 26^{th} Rev./National Formulary, 21^{st} Ed., 2002, <724> Drug Release, Delayed-Release (Enteric-Coated) Articles—General Drug Release Standard, 2160-2161; Pharmaceutical Dosage Forms and Drug Delivery Systems, H. C. Ansel, L. V. Allen, Jr., N. G. Popovich (Lippincott Williams & Wilkins, pub., 1999), Modified-Release Dosage Forms and Drug Delivery Systems, 223, 231-240).

The present invention provides an oral pharmaceutical dosage form including rasagiline and that is a tablet provided with an enteric coating.

The present invention also provides an oral pharmaceutical dosage form including rasagiline and that is a tablet that is formulated with an acid-resistant excipient other than a coating. The acid-resistant excipient can be a diluent or, preferably, a binder or disintegrant. Acid-resistant diluents and acid-resistant disintegrants are chosen, and pharmaceutical formulations containing them compressed into dosage forms, so that the dosage form (e.g. tablet) resists break-up in the stomach, thereby inhibiting or retarding release of the active pharmaceutical ingredient in the stomach. The dosage forms of this embodiment can have an enteric coating.

Acid-resistant diluents and disintegrants do not swell or dissolve to a significant extent at pH less than about 6.

The present invention also provides an oral pharmaceutical dosage form including rasagiline and that is a capsule having particles, such as spheres, microparticles, nanoparticles, or pellets of materials such as sugars, alcohols, polyols, or cellulosics, the particles having a coating of rasagiline and a further coating that is an enteric coating. Capsules containing enteric coated rasagiline pellets are useful to overcome the obstacles in treating patients with Parkinson's disease or related disorders who experience delayed gastric emptying. The small size of the enteric coated pellets can easily pass through the stomach and into the intestine. This limits the risk of prolonged exposure to acidic environment which may cause standard enteric coated tablets to fail, thereby releasing rasagiline prematurely in the stomach. Thus, release of the active pharmaceutical ingredient in the stomach is inhibited or retarded.

For some patients, however, the acid resistance of enteric coatings may be compromised by being exposed to acid for extended periods especially when being exposed to mechanical forces caused by the natural churning of the stomach. The enteric coat may start to leak or may fail altogether.

Thus, the present invention also provides an oral pharmaceutical dosage form including rasagiline, useful for treating patients with Parkinsonism, fabricated such that a core tablet is coated with an enteric coating before it is sheathed in an annular body. The enteric coating serves to inhibit or retard drug release in the stomach and allow drug release to commence in the small intestine. The dosage form of this embodiment of the present invention can be made as described in published U.S. Patent Application 2004/0052843, hereby incorporated by reference in its entirety.

The core tablet has first and second opposed surfaces and a circumferential surface. “Sheathing” means that the annular body encircles the core tablet and is in contact with the core tablet about its circumferential surface, but leaves opposed surfaces of the core tablet substantially exposed.

The annular body can be formed of any powdered or granular pharmaceutically acceptable excipients and can itself include and active pharmaceutical ingredient. In particular, the annular body can include diluents, binders, disintegrants, glidants, lubricants, flavorants, colorants and the like. Powdering and granulation with conventional excipients and the techniques for forming compressed bodies therefrom with given characteristics in terms of friability, hardness and freedom from capping is well within the knowledge of those skilled in the art of tableting.

Preferred excipients for forming the annular body include hydroxypropyl cellulose (e.g., Klucel™), hydroxypropyl methylcellulose (e.g., Methocel™), microcrystalline cellulose (e.g., AvicelT™), starch, lactose, sugars, polyvinylpyrrolidone (e.g., Kollidon™, Plasdone™), calcium phosphate, and MicrocelLac100™ (a 25:75 mixture of microcrystalline cellulose and lactose).

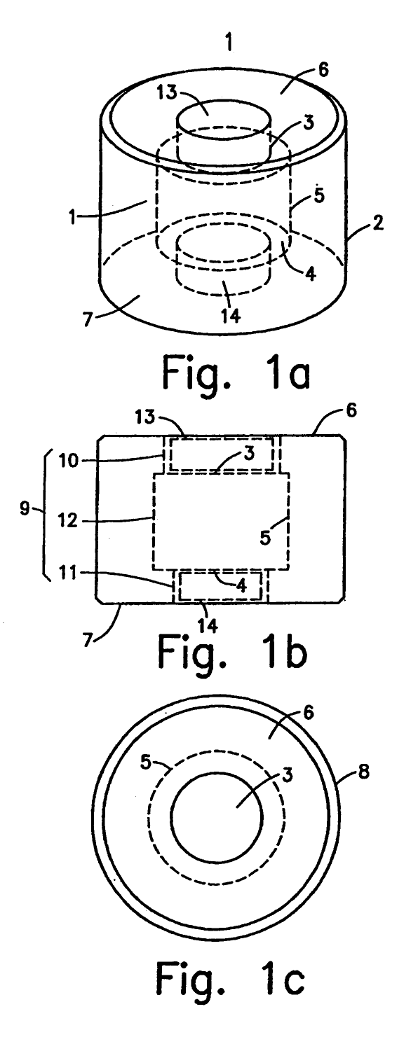

In the embodiment illustrated in FIG. 1, core tablet 1 containing rasagiline is recessed in the annular body 2. Core tablet 1 has opposed first and second surfaces 3 and 4 and an outer circumferential surface 5 extending between the opposed surfaces. Core tablet 1 is preferably cylindrical or disk shaped for ease of manufacture, but need not be so. In a dosage form for administration to humans, the maximum distance across either of the opposed surfaces 3 or 4 is preferably from about 2 mm to about 12 mm, more preferably from about 4 mm to about 7 mm, most preferably about 5 mm. Opposed surfaces 3 and 4 can be flat, concave or convex and are preferably flat for bearing modest axial compression forces exerted by flat pressing surfaces during formation of the annular body about the core tablet.

In outer contour, annular body 2 is preferably cylindrically shaped, but it can have any cross-section, such as oval, elliptical or oblong. The outer diameter is preferably of from about 5 mm to about 15 mm, more preferably of from about 7 mm to about 12 mm, most preferably about 9 mm. The inner diameter can be any size up to about 2 mm less than the outer diameter. A narrow inner diameter less than 2 mm may slow release of rasagiline if an excipient in the annular body swells upon contact with gastric fluid. However, in some embodiments, a lower limit 0.5 mm may still be useful. Preferably, the inner diameter is 3 mm or greater.

Annular body 2 has opposed first and second annular faces 6 and 7, an outer circumferential surface 8 extending between the annular faces from their outer edges, and an inner circumferential surface 9 extending between the annular surfaces from their inner edges, thus defining an annulus.

As best seen in side view FIG. 1B, inner circumferential surface 9 of annular body 2 consists of three longitudinal (axial) segments. First and second segments 10 and 11 are terminal and do not contact the sides of the core tablet. They are separated by an internal third segment 12 that contacts the outer circumferential surface 5 of core tablet 1. Opposed surfaces 3 and 4 of the core tablet are therefore recessed from annular faces 6 and 7 of the annular body. Opposed surfaces 3 and 4 are preferably recessed from about 0.5 mm to about 4 mm, more preferably about 1.5 mm relative to the annular faces 6 and 7 of the annular body (said recessed distance corresponding to the length of the corresponding terminal segment). The recess depth of surfaces 3 and 4 can be the same or it can be different.

Recessing the core tablet does not significantly alter the release profile of the core tablet because a sizable portion of the surface of the core tablet is in fluid communication with the environment. However, one or both of opposed surfaces 3 and 4 can be flush with annular faces 6 and 7 of the annular body without deleterious effect when the core tablet is protected, such as by a coating.

To better apprehend the core sheathed in an annular body dosage form, it is useful to conceive of surface 3 of the core tablet and first longitudinal segment 10 as defining a first void 13. Likewise, surface 4 of the core tablet and second longitudinal segment 11 define a second void 14. Voids 13 and 14 fill with gastric fluid when the dosage form is immersed in gastric fluid after reaching the stomach. Gastric fluid passes through the voids to contact the core tablet and the drug leaves through the voids after it is dissolved. Voids 13 and 14 are preferably from about 0.5 mm to about 10 mm, more preferably from about 3 mm to about 6 mm and most preferably about 4.5 mm in width (measured parallel to first or second opposed surfaces). Drug release, therefore, does not occur by an osmotic mechanism such as occurs with pierced dosage forms made using the apparatus of U.S. Pat. No. 5,071,607.

Opposed surfaces 3 and 4 of the core tablet are preferably substantially exposed, i.e., they are not substantially covered by the annular body. “Substantially exposed” means that less than about 50% of each of the opposed surfaces is concealed or hidden from visual inspection by the annular body. Such differences may result in inner segment 12 being offset from terminal segments 10 and 11, which, themselves, can have different longitudinal cross sections, e.g., have different diameters, as depicted in FIG. 1. Alternatively, the cross section of the annulus defined by inner circumferential surface 9 can be uniform throughout its length. Although a portion of opposed surfaces 3 and 4 can be concealed by the annular body that is not necessarily the case.

Both the core tablet and the annular body may be formed into any suitable shape, as the rate of release of rasagiline is determined by the formulation and shape of the core tablet, not by diffusion through the annular body. Specific shapes can be achieved by use of specifically designed punches. Preferably the core tablet and the annular body are cylindrical in shape. The exposed surfaces of the core tablet may be of any suitable shape. Preferably, the exposed surfaces of the core tablet are circular or oval.

The shape of one of the portions can be changed without adjusting the formulation. For instance, the powder or granular material may be pressed around the core tablet into a body having an oval cross-section rather than a circular cross-section to achieve a faster rate of release (resulting from increased surface area). In addition, the core tablet may have a hole extending from one axial face to the other in order to increase the surface and thereby increase the release rate. The release rate can be further controlled through changes to the diameter of the hole.

In all of its aspects, the present invention provides an oral pharmaceutical dosage form useful for treating a condition selected from the group consisting of: Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, and affective illness, but with a reduced risk of peripheral MAO inhibition that is typically associated with administration of rasagiline with known oral dosage forms.

Specific examples of pharmaceutical acceptable carriers and excipients that may be used to formulate oral dosage forms of the present invention are described, e.g., in U.S. Pat. No. 6,126,968 to Peskin et al., issued Oct. 3, 2000. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

As used herein, “substantial” release of rasagiline refers to a greater than 50%, 60%, 70%, 80% or 90% release of rasagiline.

This invention will be better understood from the Experimental Details which follow. However, one skilled in the art will readily appreciate that the specific methods and results discussed are merely illustrative of the invention as described more fully in the claims which follow thereafter.

Experimental Details

### EXAMPLE 1

### Enteric Coated Tablets

TABLE 1

| Tablet A | mg/tablet |
|---|---|
| Mannitol | 37.56 |
| Aerosil | 0.6 |
| Rasagiline Mesylate | 0.78 |
| Starch NF | 42.84 |
| Starch 1500 | 10 |
| Eudragit L30-D55 | 50 |
| Triethyl citrate | 5 |
| Talc | 2 |
| Stearic Acid | 2 |

All excipients except for Eudragit L-30 D-55 (methacrylic acid-ethyl acrylate copolymer (1:1) dispersion 30 percent) and triethyl citrate were mixed and granulated with water and compressed into tablets. Triethyl citrate and water were homogenized, and Eudragit was added to the homogenized mix to obtain a dispersion that contained 54% water. The tablets were sprayed with the dispersion in a Glatt Coater coating pan. The inlet air temperature was 55° C., the outlet air temperature was between 40-44° C., and the spraying rate was 20 rpm. The pan speed was set to 5 rpm.

The tablet dissolution profile was analyzed using United States Pharmacopeia method <724> for coated tablets. After 120 minutes in 0.1N HCl, the tablets were transferred to phosphate buffer solution.

TABLE 2

| | | % Dissolution |
|---|---|---|
| | % Dissolution | in phosphate |
| Time (min) | in HC1 | buffer |
| 5 | ND | 0 |
| 10 | ND | 0 |
| 15 | ND | 2 |
| 30 | 0 | 83 |
| 60 | 0 | ND |
| 90 | 0 | ND |
| 120 | 0 | ND |

The results listed in Table 2 shows that the enteric coated rasagiline tablets do not dissolve in acidic environment. Once transferred to a non-acidic environment, the tablets dissolve rapidly.

### EXAMPLE 2

### Enteric Coated Rasagiline Inner Core Tablet Sheathed in a Placebo Annular Body

An enteric coated core tablet was prepared as described below. The enteric coated tablet was sheathed in an annular body as described in published United States Publication No. 2004/0052843, published Mar. 18, 2004, incorporated herein above.

Core (Inner) Tablet

Rasagiline granulate: Rasagiline mesylate (40 grams) and a spray-dried mixture of lactose monohydrate and microcrystalline cellulose (75:25) (Microcelac 100™) (360 grams) were mixed in a Diosna P 1/6 high shear granulator at 380 rpm for 5 minutes. Purified water (130 grams) was added over the next minute while continuing to granulate at 380 rpm. The granulate was then massed for a further 1 minute at the same speed. The formed granulate was dried for 30 minutes in a Diosna Mini Lab fluidized bed drier to less than 1.5% volatiles at an inlet temperature of 60° C. and a fan setpoint of 50%. The volatile content was tested at 105° C. using a Sartorious MA 30 LOD tester.

The dried granulate was milled using a Quadro Comill with a screen of 1143μ. Two sublots were produced so as to have enough material for the next stage.

Tableting mixture: The milled, dry, rasagiline granulate (558.0 grams), was mixed in the dry state with Microcelac 100 USP (2049.6 grams), and Crospovidone NF (53.7 grams) in a 5 liter V mixer for 5 minutes. Magnesium stearate NF/EP (21.5 grams) was added and the V mixer operated for a further half a minute. The yield of the dry mix of powders was 2674.7 grams.

Tablet formation: The dry mix powder was pressed into tablets on a Kilian RTS 20 tablet press using 5 mm flat beveled punches. The tablets weighed an average of 75.0 mg, had a hardness of 8.7 Kp and a tablet thickness of 2.75 mm. The weight of the tablets produced was 2238.7 grams.

Enteric coating: Purified water (1044 grams) was placed in a mixing vessel. Talc (38.4 grams), and triethylcitrate (38.4 grams) were added and the mixture was stirred for 15 minutes with a magnetic stirrer. Eudragit L-30 D55™ (1279.2 grams) was added and the mixture stirred gently. The coating mixture was passed through a 150μ screen and then continually mixed gently.

Rasagiline core tablets (2238.7 grams) were placed in the drum of a Hi coater perforated pan coater and heated to 28-30° C. while the drum was turning at 7 rpm. The coating mixture was sprayed onto the tablets in the perforated pan coater turning at 12 rpm with the tablet bed maintained at 28-30° C. with the inlet air temperature set at 60° C. until an average of 6.5 mg per tablet of enteric coat had been added to the tablets. The tablets were air dried in the drum for five minutes after the spraying was halted and subsequently dried on an aluminum tray in a drying oven set at 40 degrees for 24 hours.

Annular Body

Tableting mixture: Polyethylene oxide (Polyox WSR-N-750™, 600 grams), Microcelac 100™(486 grams), ethylcellulose (Ethocel 7 cps, 600 grams) and polyvinylpyrollidone (Povidone K-30™, 300 grams) were placed in a 5 liter V mixer and mixed for 5 minutes. Magnesium stearate NF/EP (14 grams) was added and the V mixer operated for a further half a minute. The yield of the dry mix of powders was 1990.1 grams.

Tablet formation: The enteric coated rasagiline inner cores were added to the tablet feeder and the tableting mixture was added to the powder feeder of a Manesty LP39 press using the special spring loaded core rod tooling for making the annular sheathed tablets. The lower punch was flat beveled of 9 mm diameter and an inner hole (for the core rod) of 5 mm diameter. The upper punch was flat beveled of 9 mm diameter with a protrusion that was 1.2 mm tall and 5 mm diameter with slight tapering. The final tablets so formed weighed an average of 310 mg, had a hardness of 6.4 Kp and a tablet thickness of 5.4 mm.

Each tablet contained the equivalent of 1 mg rasagiline as the mesylate salt in the enteric coated inner core.

Results of measurement of the drug release from the dosage form are given below.

The tablets were tested for drug release in a United States Pharmacopeia Apparatus II in 500 ml 0.1N HCl at 37° C. and 50 rpm for 3 hours and then in phosphate buffer at a pH of 6.8 for an additional 2 hours. The concentration of rasagiline was measured by HPLC analysis. The results are given in Table 3. In parallel, the enteric coated tablets before their insertion into the annular sheath were also tested. These results are also given in Table 3.

TABLE 3
Cumulative Rasagiline release from a Dosage Form
of the Present Invention that is an Enterically Coated
Core Tablet Sheathed in an Annular Body

| | Core | Core |
|---|---|---|
| | within | without |
| Time | annular | annular |
| (hours) | % rasagiline | % rasagiline |
| 1 | 0 | 0 |
| 2 | 0 | 0 |
| 3 | 0 | 0 |
| 3.15 | 31 | 27 |
| 3.5 | 89 | 80 |
| 3.75 | 101 | 90.5 |
| 4 | 104 | 93.5 |
| 5 | 107 | 97.9 |

The results show that the enteric coating prevented the rasagiline from being released from the pharmaceutical dosage form for three hours in the acidic buffer both in the enteric coated tablet and in the annular sheath coated enteric tablet.

When transferred to a neutral buffer, the rasagiline was released in an immediate fashion. The annular sheath did not damage the enteric coating. When used in patients with gastric motility problems the annular sheath will be effective in protecting the enteric coating against mechanical forces in the gastrointestinal tract.

### EXAMPLE 3

### Rasagiline Capsules Containing Enteric Coated Particles

Particles (sugar spheres) for capsule filling were made using the ingredients listed in Table 4.

TABLE 4

| Ingredients | mg/capsule |
|---|---|
| Sucrose/Corn Starch | 121 |
| Spheres (92:8) (Suglets ® | |
| NPPharm) | |
| Rasagiline Mesylate | 3.12 |
| Polyethylene | 2.0 |
| Glycol (PEG 6000 NF) | |
| Hydroxypropyl | 8.0 |
| Methylcellulose | |
| (Pharmacoat 606) | |

PEG 6000 was mixed with water to form a solution. Rasagiline mesylate was then added and the solution was mixed. Hydroxypropyl methylcellulose was added to water, and the two solutions were combined and mixed. Suglets were placed in a Wurster fluid bed drier and the combined solution was sprayed on to the Suglets. The inlet temperature was 55° C., and the outlet temperature was between 29° C. and 47° C. The spray rate was between 8 and 16 gram/min. The airflow rate was between 50-120 m³/hour.

The particles (sugar spheres) were then coated with different amounts of enteric coating, as described in Table 5.

TABLE 5

| Ingredient | Capsule A | Capsule B | Capsule C | Capsule D | Capsule E | Capsule F |
|---|---|---|---|---|---|---|
| Eudragit L-30 | 10 | 13.33 | 20 | 26.8 | 33.53 | 42.64 |
| D-55 | | | | | | |
| (mg/capsule) | | | | | | |
| Triethyl | 1 | 1.342 | 2.01 | 2.68 | 3.353 | 4.29 |
| citrate | | | | | | |
| (mg/ | | | | | | |
| capsule) | | | | | | |
| % coating | 8 | 10 | 15 | 20 | 25 | 30 |

The percentage of coating was calculated as Eudragit weight/rasagiline coated particle weight.

Triethyl citrate and water were homogenized, and Eudragit was added to attain a dispersion which contained 45.4% water. The drug coated pellets were placed in the Wurster fluid bed drier a second time. The dispersion was sprayed at a rate of between 8 and 16 g/min. The inlet temperature was between 33° C. and 48° C., and the outlet temperature was between 25° C. and 45° C. The airflow rate was between 40 and 120 m³/hour. After coating, the enteric coated pellets were dried for 90 minutes. Six batches of enteric coated pellets were formed with different amounts of coating in each batch.

The enteric coated particles were then filled into HDP #1 capsules. The dissolution profile of the capsules batches in HCl 0.1 N, based on USP procedures, is shown in Table 6.

TABLE 6

| Capsule | | | | | | |
|---|---|---|---|---|---|---|
| F | E | D | C | B | A | Time (min) |
| ND | | | 11% | 18% | 24% | 30 |
| | | | 37% | 46% | 54% | 60 |
| | | | 50% | 57% | 66% | 90 |
| 3% | 11% | 23% | 59% | 64% | 75% | 120 |

The dissolution profile of the capsules in phosphate buffer is shown in Table 7.

TABLE 7

Capsule

| | | | | | | |
|---|---|---|---|---|---|---|
| F | E | D | C | B | A | Time (min) |

| 22% | ND | | | | 5 |
|---|---|---|---|---|---|
| 65% | | | | | 10 |
| 81% | | | | | 15 |
| 86% | ND | 89% | 89% | 98% | 30 |

The dissolution profile (results above) shows that formulation F is effective in protecting the spheres from being dissolved in the stomach, thereby eliminating cheese effect in patients who are treated with the capsules. Capsules comprising spheres as in formulation F would be effective in treating Parkinson's patients because the spheres maintain integrity in stomach-like conditions for two hours, and are easily soluble in intestine-like conditions.

### EXAMPLE 4

Particles (sugar spheres) for capsule filling were made using the ingredients listed in Table 8.

TABLE 8

| Ingredients | mg/capsule |
|---|---|
| Rasagiline Mesylate | 1.56 |
| PEG 6000 | 1.0 |
| Sucrose/corn starch spheres | 121 |
| (92:8) (Suglets ® NP Pharm) | |
| Hydroxypropyl | 4.0 |
| methylcellulose | |
| (Pharmacoat 606) | |
| Sodium Lauryl Sulfate | 2.0 |
| Eudragit L-30 D-55 | 38.27 |
| Triethyl citrate | 3.83 |

PEG 6000 was mixed with water to form a solution, and sodium lauryl sulfate was added. Rasagiline mesylate was added and the solution was mixed. Hydroxypropyl methylcellulose was added to water and the two solutions were combined and mixed. Suglets were placed in a Wurster fluid bed drier and the combined solution was sprayed on to the suglets. The inlet temperature was 55° C. and the outlet temperature was between 29° C. and 47° C. The spray rate was between 8 and 6 g/min. The airflow rate was between 50-120 m³/hour.

Triethyl citrate and water were homogenized, and Eudragit was added to attain a dispersion which contained 45.4% water. The drug coated pellets were placed in the Wurster fluid bed drier for a second time. The dispersion was sprayed at a rate of between 8 and 16 g/min. The inlet temperature was between 33° C. and 48° C. and the outlet temperature was between 25° C. and 45° C. The airflow rate was between 40 and 120 m³/hour. After coating, the enteric coated pellets were dried for 90 minutes.

The enteric coated pellets were then filled into Gelatin capsules, size 1. The dissolution profile based on USP procedures of the capsules is shown below in Table 9. The capsules were placed in in HCl 0.1 N. After 120 minutes in 0.1 N HCl, the dosage form was transferred to phosphate buffer solution.

TABLE 9

| | Dissolution |
|---|---|
| Time (in minutes) | (in percent) |
| 0 | 0 |
| 60 | 0 |
| 120 | 0 |
| 125 | 30 |
| 130 | 68 |
| 145 | 82 |
| 150 | 94 |
| 165 | 95 |
| 180 | 97 |

### EXAMPLE 5

### Rasagiline Immediate Release Reference Standard

Rasagiline immediate release reference standard tablets were prepared using the ingredients listed in Table 10.

TABLE 10

| Ingredients | mg/tablet |
|---|---|
| Rasagiline mesylate | 1.56 |
| Mannitol USP | 78.84 |
| Colloidal Silicon Dioxide | 0.6 |
| Starch NF | 10.0 |
| Pregelatinized Starch NF/EP | 10.0 |
| Stearic Acid NF/EP | 2.0 |
| Talc USP/EP | 2.0 |

Rasagiline mesylate, mannitol, half of the colloidal silicon dioxide, starch and pregelatinized starch were mixed in a Diosna P-800 mixer for about 5 minutes. Water was added and the mixture was mixed further. The granulate was dried and the remainder of the colloidal silicon dioxide was added. The granulate was ground in a Frewitt mill and stearic acid and talc were added. The granulate was mixed for five minutes in a tumbler and was tableted.

### EXAMPLE 6

### Plasma Concentration of Rasagiline and Aminoindan after Administration

Part 1:

Delayed release capsules were prepared as in Example 4. Immediate release tablets were prepared as a reference standard as in Example 5.

A single dose, crossover comparative PK study was performed in 12 healthy male volunteers in the fasting state. Each patient was administered a delayed release formulation in the form of 2 capsules as described in Example 4, and an immediate release formulation in the form of 2 tablets as described in Example 5. There was a separation of at least one week between the administrations of the two formulations.

Plasma concentrations of rasagiline and of its active metabolite, 1-aminoindan, were measured at the following times (in hours): 0.00, 0.08, 0.17, 0.33, 0.67, 1.00, 1.33, 1.67, 2.00, 2.33, 2.67, 3.00, 3.50, 4.00, 5.00, 6.00, 8.00, 12.00, and 24.00.

The results of the study are shown in tables 11 and 12 below:

TABLE 11

| Rasagiline | | | | | | |
|---|---|---|---|---|---|---|
| PK | Tₘₐₓ | | Cₘₐₓ | | AUC₀₋ₜ | |
| parameters | h | SD | ng/ml | SD | h * ng/ml | SD |
| Delayed | 1.83 | 1.33-4.00 | 6.4 | 1.7 | 7.8 | 1.9 |
| release | | | | | | |
| capsules | | | | | | |
| Immediate | 0.33 | 0.33-0.67 | 11.4 | 3.1 | 7.1 | 2.5 |
| release | | | | | | |
| formulation | | | | | | |

TABLE 12

| 1-Aminoindan | | | | | | |
|---|---|---|---|---|---|---|
| PK | Tₘₐₓ | | Cₘₐₓ | | AUC₀₋ₜ | |
| parameters | h | SD | ng/ml | SD | h * ng/ml | SD |
| Delayed | 2.83 | 1.33-6.00 | 2.72 | 0.72 | 32.0 | 11.3 |
| release | | | | | | |
| capsules | | | | | | |
| Immediate | 1.17 | 0.67-3.17 | 2.96 | 0.68 | 29.1 | 8.8 |
| release | | | | | | |
| formulation | | | | | | |

Cₘₐₓ is the mean maximum measured plasma concentration.
Tₘₐₓ is the mean time at which the maximum concentration was measured.
AUC₀₋ₜ is the mean area under the concentration-time curve from time zero (predose) to the time of the last quantifiable concentration. This measurement was calculated using a linear trapezoidal method.

Part 2:

Delayed release tablets were prepared as in Example 2. Immediate release tablets were prepared as a reference standard as in Example 5. A single dose, crossover comparative PK study was performed in 11 healthy male volunteers, in the fasting state. Each patient was administered a delayed release formulation in the form of 2 tablets as described in Example 2, and an immediate release formulation in the form of 2 tablets as in Example 5. There was a washout period of at least 21 days between the administrations of the two formulations. Plasma concentrations of rasagiline and of its active metabolite, 1-aminoindan, were measured at the following times (in hours): 0, 0.08, 0.17, 0.33, 0.75, 1, 1.5, 2, 2.5, 3, 4, 5, 6, 8, 10, 12, 18, 24, 36 and 48.

The results of the study are shown in tables 13 and 14 below.

TABLE 13

| Rasagiline | | | | | | |
|---|---|---|---|---|---|---|
| PK | Tₘₐₓ | | Cₘₐₓ | | AUC₀₋ₜ | |
| parameters | h | SD | ng/ml | SD | h * ng/ml | SD |
| Delayed | 3 | 2.00-4.00 | 7.75 | 4.07 | 9.69 | 3.48 |
| release | | | | | | |
| tablets | | | | | | |
| Immediate | 0.33 | 0.33-1.00 | 11.76 | 3.85 | 8.23 | 1.43 |
| release | | | | | | |
| tablets | | | | | | |

TABLE 14

| 1-Aminoindan | | | | | | |
|---|---|---|---|---|---|---|
| PK | Tₘₐₓ | | Cₘₐₓ | | AUC₀₋ₜ | |
| parameters | h | SD | ng/ml | SD | h * ng/ml | SD |
| Delayed | 4 | 3.00-5.00 | 2.14 | 0.49 | 25.22 | 8.61 |
| release | | | | | | |
| tablets | | | | | | |
| Immediate | 0.75 | 0.75-3.00 | 2.67 | 0.59 | 24.46 | 9.78 |
| release tablets | | | | | | |

Cₘₐₓ is the mean maximum measured plasma concentration.
Tₘₐₓ is the mean time at which the maximum concentration was measured.
AUC₀₋ₜ is the mean area under the concentration-time curve from time zero (predose) to the time of the last quantifiable concentration. This measurement was calculated using a linear trapezoidal method.

Results

In comparison to the immediate release tablets, significant delay of Tₘₐₓ of both rasagiline and 1-aminoindan is evident when the delayed release tablets or capsules were administered. By the time the delayed-release dosage form releases the rasagiline, about 3 hours after administration, the dosage form has already left the stomach and the duodenum, thereby eliminating any potential MAO inhibition in the stomach. This would eliminate any possible cheese effect associated with MAO inhibition in the stomach and the duodenum. The AUC₀₋ₜ of rasagiline and of 1-aminoindan in the delayed release formulations are similar to those of the immediate release tablets.

The Cₘₐₓ for the delayed release dosage forms was lower than the Cₘₐₓ in the immediate release tablets.

1. An oral pharmaceutical dosage form comprising rasagiline and adapted to retard or inhibit the release of rasagiline in the stomach.

2. The oral pharmaceutical dosage form of claim 1 that is a tablet, a capsule, or a core sheathed in an annular body.

3. The pharmaceutical dosage form of claim 2 that is a tablet.

4. The pharmaceutical dosage form of claim 2 comprising an enteric coating.

5. The pharmaceutical dosage form of claim 2 that is a core sheathed in an annular body.

6. The pharmaceutical dosage form of claim 5, wherein the core is a tablet.

7. The pharmaceutical dosage form of any of claim 5, wherein the annular body is an annular sheath.

8. The pharmaceutical dosage form of any of claim 6, wherein the core has an enteric coating.

9. The pharmaceutical dosage form of claim 2 that is a capsule.

10. The pharmaceutical dosage form of claim 9 comprising solid inactive particles.

11. The pharmaceutical dosage form of claim 10 wherein the particles are spheres, microparticles, nanoparticles or pellets made by spheronization, or a mixture thereof.

12. The pharmaceutical dosage form of claim 11 wherein the particles are spheres.

13. The pharmaceutical dosage form of claim 10, wherein the particles are comprised of sugars, alcohols, polyols or celluloses, or mixtures thereof.

14. The pharmaceutical dosage form of claim 13 wherein the particles are comprised of sugars.

15. The pharmaceutical dosage form of claim 10, wherein the solid inactive particles comprise rasagiline.

16. The pharmaceutical dosage form of claim 15, wherein the particles are coated with rasagiline.

17. The pharmaceutical dosage form of claim 10, wherein the particles comprise an enteric coating.

18. The pharmaceutical dosage form of claim 4, wherein the enteric coating comprises polymeric methacrylate.

19. The pharmaceutical dosage form of claim 18, wherein the polymeric methacrylate is methacrylic acid-ethyl acrylate copolymer (1:1) dispersion 30 percent.

20. The pharmaceutical dosage form of claim 4, wherein the enteric coating further comprises a plasticizer.

21. The pharmaceutical dosage form of claim 20 wherein the plasticizer is triethyl citrate.

22. The oral pharmaceutical dosage form of claim 1, wherein, upon administration to a patient, the rasagiline is released substantially in the intestinal tract.

23. The oral pharmaceutical dosage form of claim 22, wherein the rasagiline is released substantially in the small intestine.

24. The pharmaceutical dosage form of claim 1 having a rasagiline release profile in a United States Pharmacopoeia Apparatus II of less than 10% dissolution in 500 ml 0.1N HCl at 37° C. and 50 rpm after 3 hours, and more than 90% dissolution in phosphate buffer at a pH of 6.8 after an additional 2 hours.

25. A method of treating a patient suffering from Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, or an affective illness, comprising administering to the patient the oral dosage form of claim 1.

26. The method of claim 25 for treating a patient suffering from Parkinson's disease.

27. The method of claim 26, wherein the patient presents impaired gastric motility.

28-33. (canceled)
